(19) United States
(12) Patent Application Publication
(10) Pub. No.: US 2009/0062827 A1
(43) Pub. Date: Mar. 5, 2009

(54) VACUUM-BASED METHOD FOR OBSTRUCTION OF UTERINE ARTERIES TO TREAT UTERINE FIBROIDS

(76) Inventors: Peter Meier, Hamburg (DE); Burkhard Peters, Wattenbeck (DE); Axel Hentrich, Hamburg (DE)

Correspondence Address: GREENBERG TRAURIG, LLP, 200 PARK AVE., P.O. BOX 677, FLORHAM PARK, NJ, 07932, US

(21) Appl. No.: 11/848,787

(22) Filed: Aug. 31, 2007

(51) Int. Cl. A61B 17/08

(52) U.S. Cl. 606/158

(57) ABSTRACT

A device for degenerating a fibroid comprises a receptacle sized and shaped so as to receive a uterine artery therein and a vacuum source connected to the receptacle. The vacuum source is capable of creating a vacuum sufficient to draw a uterine artery into the receptacle, and to fold it in a manner that substantially obstructs blood flow therethrough. In a method of degenerating a fibroid, the aforesaid device is placed near a uterine artery, and a vacuum is applied to draw the uterine artery into the receptacle so that the uterine artery folds shut, thereby obstructing blood flow through the artery. The vacuum is applied for a time that is sufficient to degrade or kill the fibroid, after which the vacuum is released.

### FIELD OF THE INVENTION

This invention relates, generally, to the treatment of uterine fibroids by obstruction of the uterine arteries. More specifically, it relates to the use of mechanical instruments to block the flow of blood through the arteries.

### BACKGROUND OF THE INVENTION

Uterine leiomyomas (i.e., fibroids) are extremely common benign tumors, which are located primarily within the uterine muscle (i.e., intramural fibroids), the uterine cavity (i.e., submucosal fibroids) or on the serosal surface of the uterus. Such fibroids occur in approximately 20% to 30% of women older than 30 years of age. Medical treatment is usually sought when the fibroids are associated with menorrhagia, pelvic pain or urinary symptoms, or when they are suspected to be the cause of infertility. Treatment options include medical therapy and various types of surgical intervention.

Hysterectomy is considered to be the definitive surgical treatment for those women who no longer wish to maintain their fertility. Though effective, this method has a number of undesirable characteristics. First is the mortality rate for this procedure, which is approximately 30 times as great as the mortality rate for women who have not had hysterectomies. Further adverse effects of hysterectomies include damage to adjacent organs, including removal of the ovaries, lengthy hospital stays and periods of recovery, and an increased likelihood of cardiac arrest, decreased sexual pleasure, and increases in depression or anxiety. Surgical removal of fibroids without hysterectomy, by any surgical method, presents a risk of recurrence of fibroids or, more often, failure to observe existing fibroids or misidentification of the fibroids that are causing adverse symptoms.

It has been established that fibroids can be treated by non-surgical therapies involving the temporary obstruction of the blood flow within the arteries transporting blood into the uterus. One example of such a treatment is uterine artery embolization (UAE). UAE involves the injection of tiny particles of polyvinyl alcohol (PVA) through blood vessels to block the arteries supplying blood to the fibroids. This blockage of the blood supply causes degeneration of the fibroids leading to their death. However, UAE is performed by radiologists who, typically, are unfamiliar with practices of gynecological care. As of now, UAE's are performed in radiology suites, which have high installation and operational costs and which, therefore, are generally restricted to major medical centers. Also, however UAE is practiced, the movement of the PVA particles is flow-directed and their distribution is not limited to the arteries that supply the fibroids, but may affect blood flow to other areas of the uterine tissue or to the ovaries.

There exists a need for devices and methods that can be used to temporarily obstruct the flow of blood to fibroids. The devices should be relatively inexpensive and simple to apply, and should allow the physician to control the degree by which blood flow is reduced. Various devices and methods for obstructing the uterine arteries have been disclosed in the prior art:

U.S. Pat. No. 6,254,601 discloses methods for penetrating the wall of the vaginal vault near the uterine artery with devices that sense the locations of the anatomical structures and occlude the uterine artery. A number of methods and devices are disclosed. These disclosures are also presented in U.S. Pat. Nos. 6,602,251 and 6,764,488.

U.S. Pat. No. 6,550,482 discloses a clamp for temporarily obstructing the uterine artery. The clamp stretches the wall of the vaginal vault around the artery and applies pressure to stop blood flow.

U.S. Patent Publication No. 2002/0165579 discloses a compression device for distending the wall of the vaginal vault and thus compressing the uterine artery. Doppler ultrasound techniques are used to locate the uterine artery and sense when blood flow has stopped.

U.S. Patent Publication No. 2002/0183771, discloses a compression device that clamps around the uterine artery and the vaginal wall to stop blood flow.

U.S. Patent Publication No. 2002/0188306 discloses a forceps-type clamp that is inserted into the vagina and clamps around the uterine artery and the vaginal wall. Ultrasound sensors are placed on the ends of the clamp to allow location of the uterine artery and sense blood flow. Similar forceps-type clamps are described in a number of other references.

U.S. Patent Publication No. 2002/0124853 is directed to a method of temporarily obstructing blood flow through the uterine artery for a set period of time, then re-establishing blood flow through the artery. A forceps-type clamp is used to compress the artery from both sides.

U.S. Patent Publication No. 2004/0092979 discloses a device with paddles that are used to distend the wall of the vaginal vault around the uterus, thus compressing both uterine arteries at the same time.

U.S. Patent Publication No. 2003/0120286 discloses a clip for encircling and compressing a body lumen, of which a uterine artery is one example.

U.S. Patent Publication No. 2004/0097962 discloses constriction devices that can be deployed to distend the vaginal wall around the uterus and thus obstruct the uterine arteries.

U.S. Patent Publication No. 2005/0113852 discloses a non-invasive vacuum device for applying vacuum to the cervix thereby distending the vaginal wall around the uterus so as to obstruct the uterine arteries.

### SUMMARY OF THE INVENTION

The invention, in general, relates to a device and method for degenerating a fibroid by obstructing the flow of blood through a uterine artery. In general, the device comprises a receptacle sized and shaped to receive a uterine artery therein and a vacuum source connected to the receptacle. In one embodiment, the receptacle has a hollow cone-like portion that is hooded so as to form a gap that is sized and shaped such that a uterine artery may pass through the gap across the wide end of the cone-like portion. The receptacle is sized and shaped such that it may be inserted through an incision in the vaginal wall of a female patient and maneuvered into close proximity with the patient's uterine artery. When used for degenerating a fibroid, the embodiment creates, in its receptacle, a vacuum that is sufficient to draw a uterine artery into the receptacle and to fold it in such a manner that the flow of blood through the artery is substantially obstructed. The vacuum is maintained for a long enough time to degrade the fibroid.

It should be understood that the embodiment described above is merely exemplary and that additional embodiments may be realized that are within the scope of the invention. The invention is further described in the Detailed Description of the Invention presented below.

### BRIEF DESCRIPTION OF THE DRAWINGS

For a more complete understanding of the present invention, reference is made to the following detailed description of the present invention considered in conjunction with the accompanying drawings, in which:

FIG. 1 is an illustration of a vacuum device according to an embodiment of the present invention.

FIG. 2 is a cross-sectional view, taken generally along section line II-II in FIG. 1, of the vacuum device of FIG. 1, which has been connected to a vacuum source.

FIG. 3 is an illustration of the vacuum device of FIG. 1 penetrating the vaginal wall in proximity to a uterine artery.

FIG. 4 illustrates the device of FIG. 1 after having engaged the uterine artery.

FIG. 5 is a larger scale illustration of the vacuum device of FIG. 1 as it engages the uterine artery.

FIG. 6 is an illustration of the vacuum device and uterine artery of FIG. 5 during the application of vacuum.

### DETAILED DESCRIPTION OF THE INVENTION

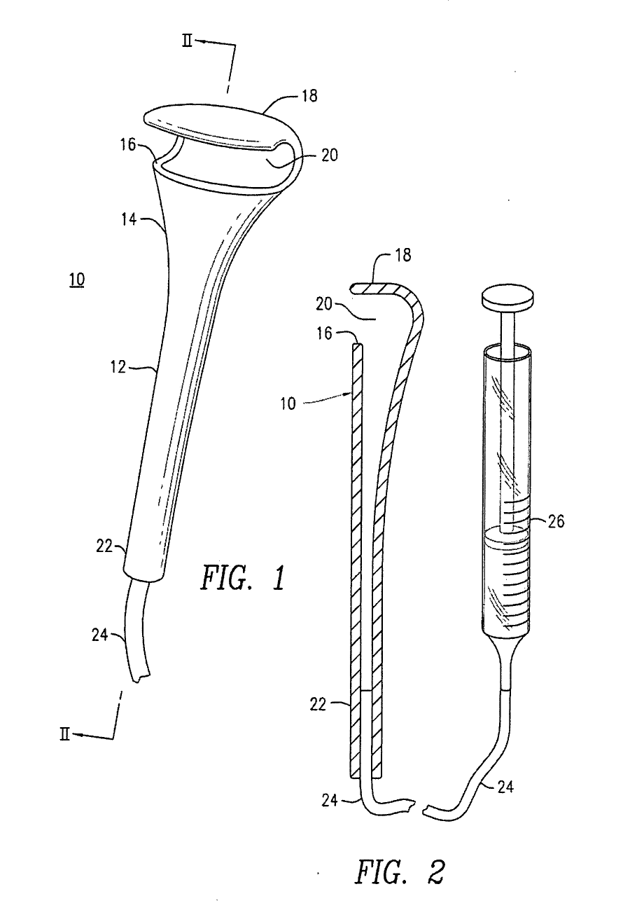

FIG. 1 illustrates a hook-like vacuum device 10 which operates directly on a blood vessel. The hook-like device 10 has a cylindrical body 12 that widens into a cone-like portion 14 having a wide end 16 that is open. A part 18 of the cone-like portion 14 extends past the wide end 16 and is folded back over the wide end 16, defining a gap 20 into which a uterine artery (not shown) may fit.

As seen in FIG. 2, the entire hook-like device 10 is hollow and is penetrated at its closed end 22 by a flexible tube 24. The flexible tube 24 may be connected to a vacuum source, such as a syringe 26 or a vacuum pump (not shown). Preferably, the hook-like device 10 is made of a rigid polymeric material. The exterior of the cylindrical body 12 and/or the cone-like portion 14 may be roughened to increase friction at the surface of the hook-like device 10 or improve ease of handling the hook-like device 10.

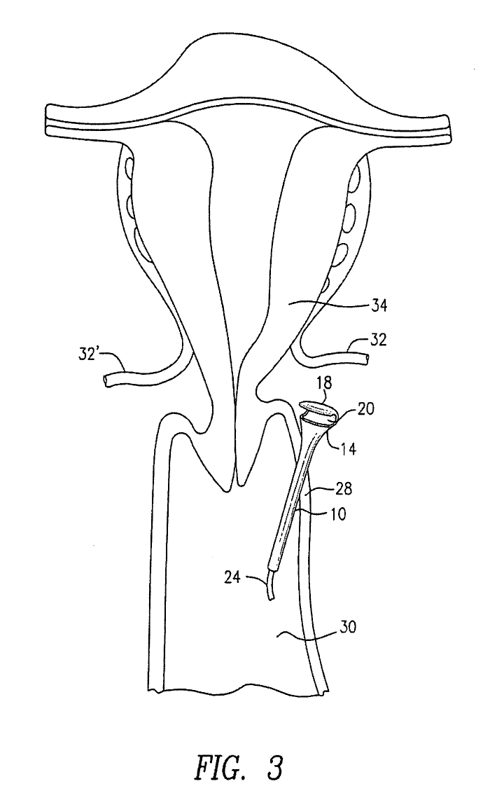

Application of the hook-like device 10 is illustrated in FIGS. 3 through 6. Referring to these figures, the portions of the female anatomy pertinent to the following discussion include the wall 28 of the vaginal vault 30, the uterine arteries 32, 32′, and the uterus 34.

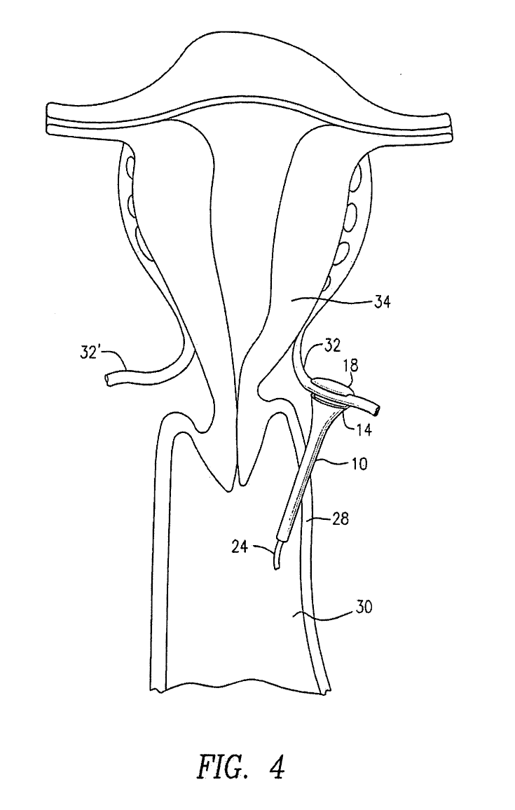

First, an incision (not shown) is made in the wall 28 of the vaginal vault 30 to expose the uterine artery 32, and the artery 32 is dissected. The cone-like portion 14 of the hook-like device 10 is inserted through the incision, as seen in FIG. 3, and maneuvered over the uterine artery 32 so as to snag the artery 32 in the gap 20, as seen in FIG. 4. The hook-like device 10 may be put into position using a forceps (not shown) or other device capable of releasably gripping the hook-like device 10.

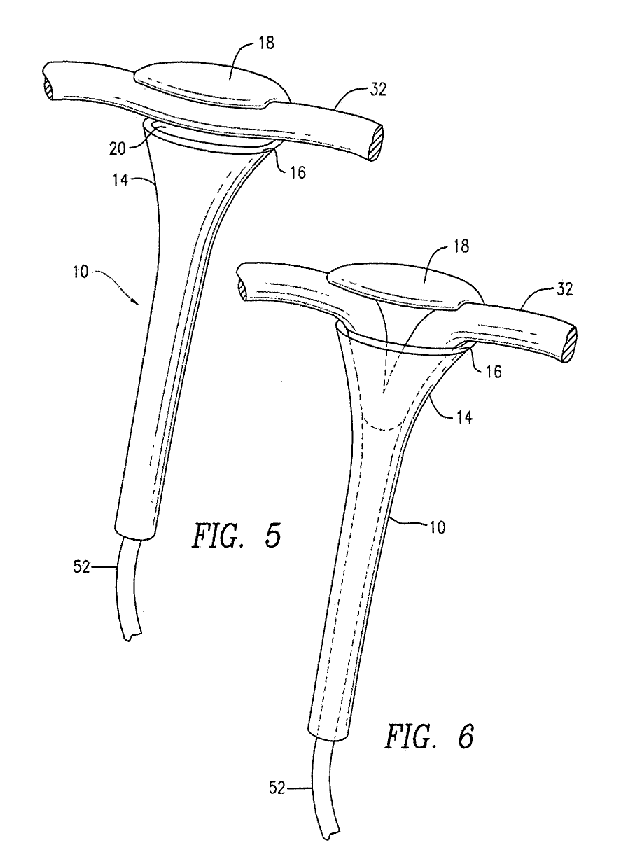

FIG. 5 shows the uterine artery 32 ensnared in the gap 20 before vacuum is applied. As vacuum is applied to the hook-like device 10, the uterine artery 32 is pulled into the cone-like portion 14, until the uterine artery 32 is pinched closed, as shown in FIG. 6, thus obstructing the flow of blood to the uterus 34.

Vacuum is applied for the length of time needed to degenerate or kill one or more fibroids without killing adjacent tissues. Preferably, blood flow through the uterine artery 32 is blocked for about 6 to 8 hours, after which time the vacuum is released, allowing the uterine artery 32 to be released from the cone-like section 14. The hook-like device 10 should be shaken gently to free the uterine artery 32 from the gap 20, allowing normal blood flow to resume. When the uterine artery 32 has been freed from the gap 20, forceps, or other suitable implements, may used to retract the hook-like device 10 through the incision and remove it from the vaginal vault 30.

During the procedure, it is important that the position of the gap 20 be known relative to the uterine artery 32, so that the hook-shaped device 10 is not mistakenly applied to another blood vessel or to the ureter (not shown). The position of the hook-like device 10 relative to the uterine artery 32 may be determined by any of a number of imaging techniques and/or techniques for monitoring the flow of blood through blood vessels. Appropriate sensors for imaging and/or blood flow monitoring include blood flow sensors, sound sensors, pressure sensors, or electromagnetic radiation sensors (e.g., X-ray detectors). Sensors may be mounted on the hook-like device 10, on the forceps or other tool used to place or remove the device 10, or on implements temporarily attached to the device 10 during insertion. Since any sensor that is used will have associated wiring it is preferable to use an implement that can be removed or detached after the device 38 is applied to the uterine artery 32. In the absence of such an implement, the patient may move about with the hook-like device 10 in place.

Techniques that may be used include direct visual examination, abdominal ultrasound, Doppler ultrasound, X-ray detection, sound detection, and angiography. Direct visual examination is the preferred technique. Ultrasound techniques are also of value, because they are reliable, real-time techniques for visualizing the position of the arterial clip in relation to the uterine artery while the procedure is underway. Doppler ultrasound techniques are beneficial, because they can also be used to determine when blood flow ceases or is restored. Optical fibers may also be used to illuminate the organs, and transmit images to an optical viewer. Adaptations of suitable techniques for use with the hook-like device 10 will be apparent to a person skilled in the application of such techniques to surgical procedures.

It should be understood that the embodiments described herein are merely exemplary and that a person skilled in the art may make many variations and modifications thereto without departing from the spirit and scope of the present invention. All such variations and modifications, including those discussed above, are intended to be included within the scope of the invention, which is described, in part, in the claims presented below.

We claim:

1. A device for degenerating a fibroid, comprising a receptacle sized and shaped so as to receive a uterine artery therein and a vacuum source connected to said receptacle, said vacuum source capable of creating a vacuum sufficient to draw the uterine artery into said receptacle and to fold it in a manner that substantially obstructs blood flow therethrough.

2. The device of claim 1, wherein said receptacle includes a hollow cone-like portion having a wide end which is open, and wherein said vacuum source is pneumatically connected to said cone-like portion.

3. The device of claim 2, wherein said device is sized and shaped so as to be insertable into a vaginal vault of a female patient.

4. The device of claim 3, wherein said cone-like portion is formed from a substantially rigid material.

5. The device of claim 3, wherein said device is sized and shaped so as to be insertable through an incision in a vaginal wall of a female patient and maneuverable into close proximity with a uterine artery of the patient.

6. The device of claim 5 wherein said wide end of said cone-like portion is hooded so as to form a gap sized and shaped such that a uterine artery may pass closely therethrough across said wide end of said cone-like portion.

7. The device of claim 6, wherein said vacuum source is capable of creating a vacuum sufficient to draw a uterine artery lying in said gap into said cone-like portion where it is pinched shut.

8. The device of claim 1, wherein said vacuum source is a syringe.

9. The device of claim 1, wherein said vacuum source is a vacuum pump.

10. A method of degenerating a fibroid, comprising the steps of:
providing a device having a receptacle sized and shaped so as to receive a uterine artery therein; and
creating a vacuum in said receptacle sufficient to draw a uterine artery into said receptacle and to fold it in a manner that substantially obstructs blood flow therethrough.

11. The method of claim 10, wherein said vacuum is directly applied to the uterine artery.

12. The method of claim 10, wherein said vacuum is maintained long enough to hold the uterine artery closed for a time that is sufficient to degrade the fibroid.

13. The method of claim 12, further comprising the further steps of:
making an incision in the wall of the vaginal vault near the uterine artery;
dissecting the uterine artery; and
inserting the device through the incision until the receptacle receives the uterine artery, said further steps being performed before said vacuum is created.
